(19) United States
(12) Patent Application Publication
(10) Pub. No.: US 2022/0080190 A1
(43) Pub. Date: Mar. 17, 2022

(54) DEVICES, SYSTEMS, AND METHODS FOR IDENTIFYING A TARGET LOCATION FOR IMPLANTATION OF A PACING LEAD TO TREAT HEART FAILURE

(71) Applicant: Ghassan S. Kassab, La Jolla, CA (US)

(72) Inventor: Ghassan S. Kassab, La Jolla, CA (US)

(21) Appl. No.: 17/476,425

(22) Filed: Sep. 15, 2021

Related U.S. Application Data
(60) Provisional application No. 63/078,878, filed on Sep. 15, 2020.

(51) Int. Cl. A61N 1/05; A61N 1/375; A61N 1/362; A61M 25/04

(52) U.S. Cl. CPC A61N 1/0592; A61N 1/37512; A61M 2230/04; A61M 25/04; A61M 2210/125; A61N 1/3622

(57) ABSTRACT

Devices, systems, and methods for identifying a target location for implantation of a pacing lead to treat heart failure. The present disclosure includes disclosure of a method, comprising the steps of inserting at least part of a suction catheter into a blood vessel of a patient and advancing the at least part of the suction catheter to a heart, suctionally engaging a tissue of the heart using the suction catheter, positioning a pacing lead from a lumen of the suction catheter into the tissue of the heart at a first location, and operating the pacing lead in attempt to sense and confirm the presence of an indicator of the heart.

### PRIORITY

The present application is related to, and claims the priority benefit of, U.S. Provisional Patent Application Ser. No. 63/078,878, filed Sep. 15, 2020, the contents of which are incorporated herein by reference and directly in their entirety.

### BACKGROUND

Heart failure (HF) is a nationwide epidemic with over 6 million afflicted patients and 600,000 new patients diagnosed each year. Treatments of HF include medication and device therapies. Many HF patients are presented with left bundle branch block (LBBB) morphology and mechanical dyssynchrony which are strong predictors of cardiovascular mortality. For patients who have prolonged QRS duration (>150 ms), cardiac resynchronization therapy (CRT) is used to restore normal activation pattern in the heart. While CRT has emerged as a powerful treatment for HF, 30% of patients still do not improve after therapy (non-responders). Improvement of responder rate therefore remains a crucial clinical challenge and in need of further innovation.

To address the issue of CRT non-responders and to improve the treatment conduction disorders in HF, there have been new developments in pacing therapy (e.g., HIS bundle pacing, left ventricular (LV) endocardial pacing, LV septal pacing, and the like). The goal of each of these approaches is to restore a normal activation pattern in the heart. Among these new approaches, HIS bundle pacing arguably produces the most physiological pattern of ventricular activation via the native His-Purkinje System (HPS). Recent studies have also shown that HIS bundle pacing produces an LV activation time map that closely resembles the intrinsic activation pattern, a greater reduction in QRS duration and improvement in hemodynamic response (vs. CRT), preserving coronary flow (vs. right ventricular, RV, pacing) as well as improving long-term LV function.

Despite these initial promising results, there is a lack of device tools to enable systematic and reproducible HIS pacing or septal pacing to recruit the natural Purkinje system. The identification of the HIS bundle in the right atrium or the Purkinje fibers in the septum is not trivial and there are currently no dedicated tools to enable identification and consequent engagement/pacing of these systems.

### BRIEF SUMMARY

The present disclosure includes disclosure of a method, comprising the steps of a) inserting at least part of a suction catheter into a blood vessel of a patient and advancing the at least part of the suction catheter to a heart, b) suctionally engaging a tissue of the heart using the suction catheter, c) positioning a pacing lead from a lumen of the suction catheter into the tissue of the heart at a first location, and d) operating the pacing lead in attempt to sense and confirm the presence of an indicator of the heart.

The present disclosure includes disclosure of a method, wherein the indicator is selected from the group consisting of a His bundle structure of the heart and purkiunje electrical morphology of the heart.

The present disclosure includes disclosure of a method, wherein the indicator comprises a His bundle structure of the heart.

The present disclosure includes disclosure of a method, wherein the indicator comprises a purkiunje electrical morphology of the heart.

The present disclosure includes disclosure of a method, wherein the step of inserting is performed by percutaneously inserting the at least part of the suction catheter into the blood vessel.

The present disclosure includes disclosure of a method, further comprising the step of e) removing the pacing lead from the tissue of the heart if the step of operating the pacing lead does not sense and confirm the presence of the indicator.

The present disclosure includes disclosure of a method, further comprising the step of f) positioning the pacing lead from into the tissue of the heart at a second location.

The present disclosure includes disclosure of a method, further comprising the step of g) repeating step d).

The present disclosure includes disclosure of a method, further comprising the steps of h)

repeating step e), and i) positioning the pacing lead from into the tissue of the heart at an additional location.

The present disclosure includes disclosure of a method, further comprising the step of j) repeating step d).

The present disclosure includes disclosure of a method, further comprising the step of j) repeating step d).

The present disclosure includes disclosure of a method, further comprising the step of k) repeating steps e), i), and d) until operation of the pacing lead senses and confirms the presence of the indicator.

The present disclosure includes disclosure of a method, further comprising the step of discontinuing suction when the pacing lead senses and confirms the presence of the indicator so that the suction catheter disengages the tissue, wherein the pacing lead remains in the tissue of the heart.

The present disclosure includes disclosure of a method, wherein the indicator comprises the His bundle structure, and wherein the method further comprises the step of pacing the heart using the pacing lead at the His bundle structure to treat heart failure.

The present disclosure includes disclosure of a method, wherein step a) is performed to advance the at least part of the suction catheter to a right ventricle of the heart, wherein the tissue comprises a septal wall of the heart, wherein the indicator comprises the purkiunje electrical morphology of the heart.

The present disclosure includes disclosure of a method, further comprising the step of pacing the heart using the pacing lead at a location in the septal wall where the purkiunje electrical morphology of the heart is detected to treat heart failure.

The present disclosure includes disclosure of a method, further comprising the steps of removing the pacing lead from the tissue of the heart if the step of operating the pacing lead does not sense and confirm the presence of the indicator, positioning the pacing lead from into the tissue of the heart at one or more additional locations, and discontinuing suction when the pacing lead senses and confirms the presence of the indicator so that the suction catheter disengages the tissue, wherein the pacing lead remains in the tissue of the heart.

The present disclosure includes disclosure of a method, wherein the indicator comprises the His bundle structure, and wherein the method further comprises the step of pacing the heart using the pacing lead at the His bundle structure to treat heart failure.

The present disclosure includes disclosure of a method, wherein step a) is performed to advance the at least part of the suction catheter to a right ventricle of the heart, wherein the tissue comprises a septal wall of the heart, wherein the indicator comprises the purkiunje electrical morphology of the heart.

The present disclosure includes disclosure of a method, further comprising the step of pacing the heart using the pacing lead at a location in the septal wall where the purkiunje electrical morphology of the heart is detected to treat heart failure.

The present disclosure includes disclosure of a device, as shown and/or described herein. The present disclosure also includes disclosure of a method, as described herein.

The present disclosure includes disclosure of a method, comprising the steps of a) percutaneously inserting at least part of a suction catheter into a blood vessel of a patient and advancing the at least part of the suction catheter to a heart; b) suctionally engaging a tissue of the heart using the suction catheter; c) positioning a pacing lead from a lumen of the suction catheter into the tissue of the heart at a first location; and d) operating the pacing lead in attempt to sense and confirm the presence of an indicator of the heart, the indicator selected from the group consisting of a His bundle structure of the heart and purkiunje electrical morphology of the heart.

The present disclosure includes disclosure of a method, further comprising the step of e) removing the pacing lead from the tissue of the heart if the step of operating the pacing lead does not sense and confirm the presence of the indicator.

The present disclosure includes disclosure of a method, further comprising the steps of f) positioning the pacing lead from into the tissue of the heart at a second location; and g) repeating step d).

The present disclosure includes disclosure of a method, further comprising the steps of h) repeating step e); i) positioning the pacing lead from into the tissue of the heart at an additional location; and j) repeating step d); and k) repeating steps e), i), and d) until operation of the pacing lead senses and confirms the presence of the indicator.

The present disclosure includes disclosure of a method, further comprising the step of e) discontinuing suction when the pacing lead senses and confirms the presence of the indicator so that the suction catheter disengages the tissue, wherein the pacing lead remains in the tissue of the heart.

The present disclosure includes disclosure of a method, wherein the indicator comprises the His bundle structure, and wherein the method further comprises the step of f) pacing the heart using the pacing lead at the His bundle structure to treat heart failure.

The present disclosure includes disclosure of a method, wherein step a) is performed to advance the at least part of the suction catheter to a right ventricle of the heart, wherein the tissue comprises a septal wall of the heart, wherein the indicator comprises the purkiunje electrical morphology of the heart.

The present disclosure includes disclosure of a method, further comprising the step of f) pacing the heart using the pacing lead at a location in the septal wall where the purkiunje electrical morphology of the heart is detected to treat heart failure.

The present disclosure includes disclosure of a method to treat heart failure, as described herein.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a distal portion of system for engaging tissue and/or delivering a pacing lead, according to an exemplary embodiment of the present disclosure;

FIG. 2 shows a distal portion of a system suctionally affixed to a mammalian tissue so to deliver a pacing lead under suction, according to an exemplary embodiment of the present disclosure; and

FIG. 3 shows a pacing lead implanted into mammalian tissue with the system retracted away from the tissue, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

An SL1-type catheter, such as described within U.S. Pat. No. 8,328,752 of Kassab et al., can be used such that upon engagement, the catheter can deploy a suction tip that can attach to any part of the heart structure. In this disclosure, the suction catheter can be used to attach to the HIS bundle structure (typically below the fossa ovalis). Once engaged, a pacing lead can be advanced through the lumen of the catheter and into the cardiac tissue for sensing/capture to confirm HIS bundle. If the expected HIS electrical morphology is not confirmed, the lead can be retracted and suction can be turned off and the device repositioned. The procedure can be repeated until HIS morphology is confirmed. Once confirmed, the lead can be left in place to pace the desired structure. If septal pacing is preferred (or if HIS pacing is not successful), the articulating suction catheter can be advanced to the RV to engage the septal wall. Suction can be applied at a given septal position and lead can be advanced into the tissue similar to above to confirm purkinje morphology. If the expected purkinje electrical morphology is not confirmed, the lead can be retracted and suction can be turned off and the device repositioned. The procedure can be repeated until purkinje morphology is confirmed. Once confirmed, the lead can be left in place to pace the LV wall through the septal engagement.

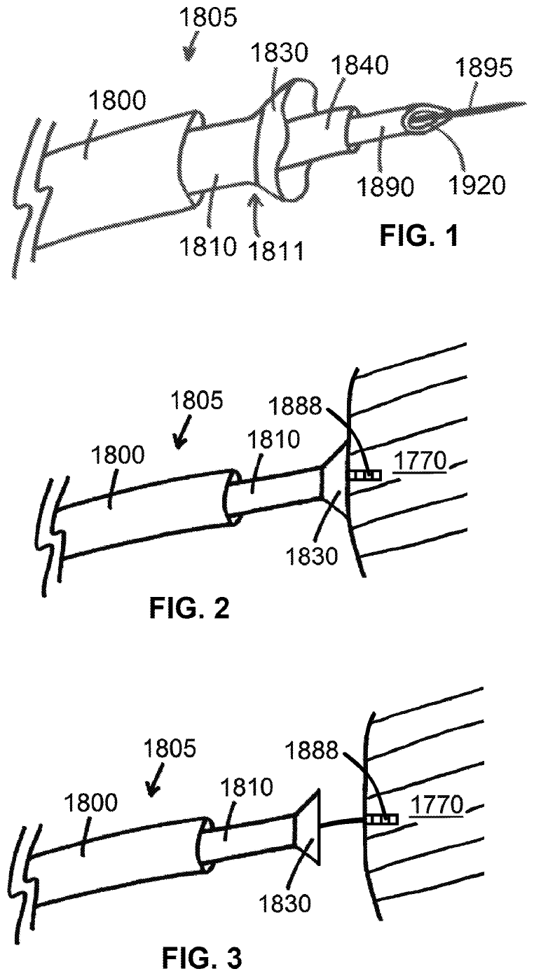

An exemplary device and related system for use in performing the various methods discussed herein are shown in FIG. 1. As shown in FIG. 1, a distal portion of an exemplary system 1805 may comprise an engagement catheter 1810 having a skirt or suction cup 1830 at or near a distal end 1811 of engagement catheter 1810. System 1805 may further comprise a sleeve 1800 positioned around portions of engagement catheter 1810 and configured for sliding movement relative to engagement catheter 1810 such that movement of sleeve 1800 relative to engagement catheter 1810 can cause skirt or suction cup 1830 to be within or external to sleeve 1800. System 1805 may further comprise a delivery catheter 1840 configured to fit within engagement catheter 1810 and configured for sliding movement relative to engagement catheter 1810. System 1805 may further comprise a needle 1890 defining a needle aperture 1920 and configured to fit within delivery catheter 1840 and/or engagement catheter 1810 and configured for sliding movement relative to delivery catheter 1840 and/or engagement catheter 1810. System 1805 may further comprise a wire 1895 configured to fit within engagement catheter 1810, delivery catheter 1840, and/or needle 1890, and configured for sliding movement relative to engagement catheter 1810, delivery catheter 1840, and/or needle 1890. Components of such exemplary system 1805 embodiments may be as described within U.S. Pat. No. 8,328,752 of Kassab et al., the contents of which are expressly incorporated herein by reference.

FIG. 2 shows a distal portion of an exemplary system 1805 of the present disclosure, with system 1805 comprising a sleeve 1800 positioned at least partially around engagement catheter 1810 having a skirt or suction cup 1830 at or near a distal end 1811 of engagement catheter 1810. Skirt or suction cup 1830 is shown as engaging tissue 1770 (which here can be cardiac tissue, such as an interatrial septum (septal wall), myocardium, a left ventricle, or other portions of cardiac tissue 1770) or other mammalian tissue. Such engagement is provided via suction through engagement catheter 1810. Suction can be provided as described within U.S. Pat. No. 8,328,752 of Kassab et al., noting that various portions of devices and/or systems disclosed within U.S. Pat. No. 8,328,752 of Kassab et al. may be used in connection with devices and/or systems of the present disclosure.

FIG. 2 also shows placement of a pacing lead 1888 within tissue 1770, which is delivered through engagement catheter 1810, such as by way of delivery catheter 1840, needle 1890, or generally within engagement catheter 1810 itself. Lead placement can be performed under suction, as referenced above, and once it is determined that the pacing lead 1888 is positioned at its desired location, engagement catheter 1810 (and other portions of system 1805, as applicable), can be retracted and removed from the patient's body, leaving pacing lead 1888 implanted into tissue 1770 (such as shown in FIG. 3).

This innovation will help optimize CRT therapy to reduce non-responder rates as well develop novel tools for new pacing therapies. This would have substantial impact on improving the treatment and reducing the cost of HF epidemic.

Cardiac Resynchronization Therapy (CRT) has emerged as a powerful treatment for HF. Approximately 30% of patients still do not improve after therapy, however, and this “non-responder” rate has remained relatively constant over the past two decades since the inception of the therapy. Improvement of the responder rate to pacing therefore remains a crucial clinical challenge. CRT pacing does not truly mirror normal activation through the Purkinje fibers, as the Purkinje fibers trigger activation initially at the apex of the heart, to result in the more hemodynamically efficient twisting contractile action present in normal cardiac function.

His and LBB pacing have therefore recently emerged as alternatives to cardiac CRT. His bundle pacing and LBB pacing arguably produce a more physiologically normal ventricular activation pattern, by utilizing the native His-Purkinje system. Recent studies have also shown LV activation time maps more closely resemble intrinsic activation patterns, a greater reduction in QRS duration (vs. CRT), and improvement in hemodynamic response (vs. CRT), preserving coronary flow (vs. RV pacing) as well as improving long-term LV function. Despite creation of specialized pacing electrodes and sheaths, however, successful placement of the His bundle pacing lead is achieved in ˜80% of cases.

As referenced herein, a method of the present disclosure comprises locating the His signal, delivering lead 1888 perpendicular to the myocardial tissue (an exemplary tissue 1770) to reach the His, and pacing to demonstrate selective His capture with acceptable pacing capture thresholds. Mapping is complicated by variability in cardiac anatomy, such as right atrium (RA) enlargement, rotation, and location introduces complexity in both locating the His and lead delivery. It is important that the His lead be advanced into tissue, ideally in a trajectory that is perpendicular to the endocardial surface, and therefore to reach the intended target given the length of lead 1888.

While various embodiments of devices, systems, and methods for identifying a target location for implantation of a pacing lead to treat heart failure have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method, comprising:
a) inserting at least part of a suction catheter into a blood vessel of a patient and advancing the at least part of the suction catheter to a heart;
b) suctionally engaging a tissue of the heart using the suction catheter;
c) positioning a pacing lead from a lumen of the suction catheter into the tissue of the heart at a first location; and
d) operating the pacing lead in attempt to sense and confirm the presence of an indicator of the heart.

2. The method of claim 1, wherein the indicator is selected from the group consisting of a His bundle structure of the heart and purkiunje electrical morphology of the heart.

3. The method of claim 1, wherein the indicator comprises a His bundle structure of the heart.

4. The method of claim 1, wherein the indicator comprises a purkiunje electrical morphology of the heart.

5. The method of claim 1, wherein the step of inserting is performed by percutaneously inserting the at least part of the suction catheter into the blood vessel.

6. The method of claim 1, further comprising the step of:
e) removing the pacing lead from the tissue of the heart if the step of operating the pacing lead does not sense and confirm the presence of the indicator.

7. The method of claim 6, further comprising the step of:
f) positioning the pacing lead from into the tissue of the heart at a second location.

8. The method of claim 7, further comprising the step of:
g) repeating step d).

9. The method of claim 8, further comprising the steps of:
h) repeating step e); and
i) positioning the pacing lead from into the tissue of the heart at an additional location.

10. The method of claim 9, further comprising the step of:
j) repeating step d).

11. The method of claim 10, further comprising the step of:
j) repeating step d).

12. The method of claim 11, further comprising the step of:
k) repeating steps e), i), and d) until operation of the pacing lead senses and confirms the presence of the indicator.

13. The method of claim 2, further comprising the step of:
discontinuing suction when the pacing lead senses and confirms the presence of the indicator so that the suction catheter disengages the tissue, wherein the pacing lead remains in the tissue of the heart.

14. The method of claim 13, wherein the indicator comprises the His bundle structure, and wherein the method further comprises the step of:
pacing the heart using the pacing lead at the His bundle structure to treat heart failure.

15. The method of claim 13, wherein step a) is performed to advance the at least part of the suction catheter to a right ventricle of the heart, wherein the tissue comprises a septal wall of the heart, wherein the indicator comprises the purkiunje electrical morphology of the heart.

16. The method of claim 15, further comprising the step of:
pacing the heart using the pacing lead at a location in the septal wall where the purkiunje electrical morphology of the heart is detected to treat heart failure.

17. The method of claim 1, further comprising the steps of:
removing the pacing lead from the tissue of the heart if the step of operating the pacing lead does not sense and confirm the presence of the indicator;
positioning the pacing lead from into the tissue of the heart at one or more additional locations; and
discontinuing suction when the pacing lead senses and confirms the presence of the indicator so that the suction catheter disengages the tissue, wherein the pacing lead remains in the tissue of the heart.

18. The method of claim 17, wherein the indicator comprises the His bundle structure, and wherein the method further comprises the step of:
pacing the heart using the pacing lead at the His bundle structure to treat heart failure.

19. The method of claim 17, wherein step a) is performed to advance the at least part of the suction catheter to a right ventricle of the heart, wherein the tissue comprises a septal wall of the heart, wherein the indicator comprises the purkiunje electrical morphology of the heart.

20. The method of claim 18, further comprising the step of:
pacing the heart using the pacing lead at a location in the septal wall where the purkiunje electrical morphology of the heart is detected to treat heart failure.
